(19) United States
(12) Patent Application Publication
(10) Pub. No.: US 2020/0051696 A1
(43) Pub. Date: Feb. 13, 2020

(54) METHOD FOR TRANSFORMING PATIENT DATA INTO IMAGES FOR INFECTION PREDICTION

(71) Applicant: KONINKLIJKE PHILIPS N.V., Eindhoven (NL)

(72) Inventors: Claire Zhao, Cambridge, MA (US); Jonathan Rubin, Cambridge, MA (US); Bryan Conroy, Garden City South, NY (US); Asif Rahman, Brookline, MA (US); Minnan Xu, Cambridge, MA (US)

(21) Appl. No.: 16/533,912

(22) Filed: Aug. 7, 2019

Related U.S. Application Data
(60) Provisional application No. 62/715,857, filed on Aug. 8, 2018.

(51) Int. Cl. G16H 50/30; G16H 10/60; G16H 50/70

(52) U.S. Cl. CPC G16H 50/30; G16H 15/00; G16H 50/70; G16H 10/60

(57) ABSTRACT

A method of determining the infection risk probability for a patient, including: encoding physiological data of the patient into a first synthetic image; encoding environmental data of the patient into a second synthetic image; determining an intrinsic probability of infection for the patient based upon the first synthetic image and the second synthetic image using a machine learning model; generating a graphical model based upon the patient and other patients based upon similarity scores between the patient and the other patients; and determining the infection risk probability for the patient based upon the graphical model and the intrinsic probability of infection for the patient and the other patients.

### TECHNICAL FIELD

Various exemplary embodiments disclosed herein relate generally to a method for transforming patient data into images for infection prediction.

### BACKGROUND

Prediction of risk of infection is critical to reducing morbidity and mortality because it allows time for adequate preparation and timely implementation of disease prevention and control measures. The inpatient setting is where various kinds of infections can be easily spread. First of all, pathogens are more prevalent in this setting because many patients already carry pathogens and the spread of pathogens are facilitated by many clinical procedures performed. In addition, patients can be easily infected by and host pathogens due to their declining immune response and general physical deterioration.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method of determining the infection risk probability for a patient, including: encoding physiological data of the patient into a first synthetic image; encoding environmental data of the patient into a second synthetic image; determining an intrinsic probability of infection for the patient based upon the first synthetic image and the second synthetic image using a machine learning model; determining patterns of infection transmission by generating a graphical model based upon the intrinsic probability of patients based upon similarity scores between the patient and the other patients; and determining the infection risk probability for the patient based upon the graphical model and the intrinsic probability of infection for the patient and the other patients.

Various embodiments are described, wherein the first synthetic image is a radar-type chart where each data parameter of the physiological data is encoded by an angle, the time and effective duration of the data parameter is encoded by the position and length of a segment on a radius, and the value of the data parameter is encoded as a gray scale value for the portion of the first synthetic image corresponding to the data parameter.

Various embodiments are described, wherein the radius encoding of the data proceeds from the center of to the outer boundaries of the circle along the radius based upon the time of the data parameter from the earliest time to the most recent time.

Various embodiments are described, wherein the second synthetic image is a circular slice-based image where each day includes the same angular extent and each environmental parameter is encoded as a slice of a day wherein the angular extent of the slice indicates the duration of the environmental parameter and the gray scale value of the slice indicated a code associated with the environmental parameter.

Various embodiments are described, wherein the radius of the slice indicates the total duration of all environmental parameters for the day.

Various embodiments are described, further including processing the first synthetic image and the second synthetic image into a predetermined number of image pixels with discrete values before determining an intrinsic probability of infection for the patient.

Various embodiments are described, further including generating a lattice representation of the of the patient facility indicating the location of the patients in the facility and the barriers separating the patients.

Various embodiments are described, wherein the graphical model includes a node for each patient and edges between each of the nodes indicating the similarity metric between each of the patients and wherein the graphical model is based upon the lattice representation.

Various embodiments are described, wherein the similarity metric between two patients is based upon the first synthetic images and the second synthetic images of the two patients.

Various embodiments are described, wherein the similarity metric between two patients is based upon the distance between the two patients based upon the lattice representation.

Various embodiments are described, wherein the similarity metric between two patients is further based upon the barriers between the two patients.

Various embodiments are described, wherein determining the infection risk probability for the patient is based on the weighted sum of the intrinsic probability of infection of the other patients where the similarity metrics are used as weights.

Further various embodiments relate to a non-transitory machine-readable storage medium encoded with instructions for deterring the infection risk probability for a patient, including: instructions for encoding physiological data of the patient into a first synthetic image; instructions for encoding environmental data of the patient into a second synthetic image; instructions for determining an intrinsic probability of infection for the patient based upon the first synthetic image and the second synthetic image using a machine learning model; instructions for generating a graphical model based upon the patient and other patients based upon similarity scores between the patient and the other patients; and instructions for determining the infection risk probability for the patient based upon the graphical model and the intrinsic probability of infection for the patient and the other patients.

Various embodiments are described, wherein the first synthetic image is a radar-type chart where each data parameter of the physiological data is encoded by an angle, the time and effective duration of the data parameter is encoded by the position and length of a segment on a radius, and the value of the data parameter is encoded as a gray scale value for the portion of the first synthetic image corresponding to the data parameter.

Various embodiments are described, wherein the radius encoding of the data proceeds from the center to the boundary of the circle based upon the time of the data parameter from the earliest time to the most recent time.

Various embodiments are described, wherein the second synthetic image is a circular slice-based image where each day includes the same angular extent and each environmental parameter is encoded as a slice of a day wherein the angular extent of the slice indicates the duration of the environmental parameter and the gray scale value of the slice indicated a code associated with the environmental parameter.

Various embodiments are described, wherein the radius of the slice indicates the total duration of all environmental parameters for the day.

Various embodiments are described, further including instructions for processing the first synthetic image and the second synthetic image into a predetermined number of image pixels with discrete values before determining an intrinsic probability of infection for the patient.

Various embodiments are described, further including instructions for generating a lattice representation of the of the patient facility indicating the location of the patients in the facility and the barriers separating the patients.

Various embodiments are described, wherein the graphical model includes a node for each patient and edges between each of the nodes indicating the similarity metric between each of the patients and wherein the graphical model is based upon the lattice representation.

Various embodiments are described, wherein the similarity metric between two patients is based upon the first synthetic images and the second synthetic images of the two patients.

Various embodiments are described, wherein the similarity metric between two patients is based upon the distance between the two patients based upon the lattice representation.

Various embodiments are described, wherein the similarity metric between two patients is further based upon the barriers between the two patients.

Various embodiments are described, wherein determining the infection risk probability for the patient is based on the weighted sum of the intrinsic probability of infection of the other patients where the similarity metrics are used as weights.

### BRIEF DESCRIPTION OF THE DRAWINGS

In order to better understand various exemplary embodiments, reference is made to the accompanying drawings, wherein:

FIG. 1 illustrates the physiological data of the patient encoded in a radar-chart based synthetic image;

FIG. 2 illustrates the people, equipment, and clinical environments that the patient comes into contact encoded in a slice-based synthetic image;

FIG. 3 illustrates a system for predicting the risk of infection for each patient;

FIG. 4A illustrates an example floor layout of a unit in a hospital;

FIG. 4B illustrates a lattice representation of the floor layout;

FIG. 5 illustrates two different computations for the physical distance between two patients; and

FIG. 6 illustrates a graphical model of the infection risk relationships between the patients.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Current solutions for infection prediction are based on analyses of hospital visits and/or pathogen genome sequence. They generally overlook patient-specific information. In addition, information from the hospital workflow are often ignored; therefore, the dynamic nature of interaction of the environment and patient are not exploited for infection prediction. Other methods predict infection outbreaks at a population level and are targeted to a large geographic area. These methods do not readily adapt to monitoring the individual patient in each hospital unit.

The embodiments described herein relate to a method for identifying the likelihood of a patient getting an infection based on their physiological status as well as the patients surrounding them and possible routes of infection transmission based upon the hospital layout. Conceptually, the method may be divided into three main stages: 1) patient data is transformed into synthetic images (stage 1); 2) a machine learning model such as a convolutional neural network (CNN) is used to predict probability of infection for each individual patient based on the synthetic images generated previously (stage 2); and 3) a graphical model of the layout of the hospital is used to detect possible routes of disease transmission, based on which the probability of infection previously obtained for each patient is adjusted (stage 3).

The method may include the following seven steps: 1) physiological data of the patient is encoded into a radar-chart based synthetic image; 2) people, equipment, and clinical environments that the patient comes into contact with is encoded into a slice-based synthetic image; 3) pre-processing, such as discretization and quantization, is performed on the previously generated synthetic images; 4) The pre-processed images are input to a CNN to predict a probably of infection for the patient; 5) the architectural layout of the hospital is transformed into a lattice representation; 6) patient similarity is defined based on information collected in steps 3 and 4; and 7) the metrics computed in steps 4 and 5 are formalized into a graphical model, based upon which the probability computed in step 4 will be adjusted. The risk probability computed in step 4 is the intrinsic risk arising from the individual patient's physiology; that computed in step 7 is the overall risk taking into consideration of the possible routes of infection transmission. The seven steps will now be described in more detail below.

First, the following variables are defined:

T=time window of information to be encoded to images;

TU=time unit where clinical measures are grouped into; and

t_{fi}=sample period for the fᵢ^{th} feature, fᵢ=1, 2, . . . , N_{f}, where N_{f} is the number of features.

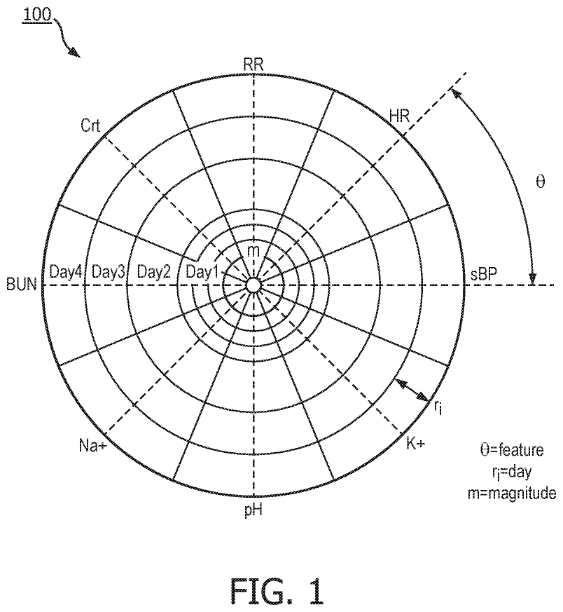

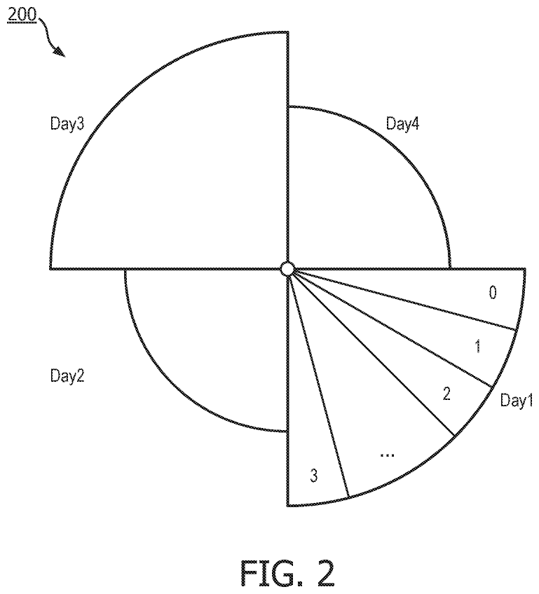

FIG. 1 illustrates the physiological data of the patient encoded in a radar-chart based synthetic image. FIG. 2 illustrates the people, equipment, and clinical environments that the patient comes into contact encoded in a slice-based synthetic image. FIGS. 1 and 2 illustrate image generation for 4 days of data (T=4 days) and each day is regarded as a Time Unit (TU=1 day).

In step 1, data describing patient physiology (e.g., vitals, labs, microbiology, etc.) is encoded in a radar chart as shown in FIG. 1. Each direction/angle, θ, encodes one feature, for example, respiratory rate (RR), heart rate (HR), systolic blood pressure (sBP), blood potassium (K⁺), pH, blood sodium (Na⁺), blood urea nitrogen (BUN), and creatinine (Crt). The value, m, of the data is encoded in a grey scale value for the portion of the image corresponding to the data. The earliest data in the time window T is kept in the inner most ring. As newer data arrives, another ring is added. Thus, the outer-most ring holds the most recent data. The thickness of the ring for each Time Unit (TUᵢ) is constant (rᵢ). Because the more recent physiological data for a particular patient is more important, the more recent information needs to be encoded by larger area, which is subsequently referred to as the area condition. As a result, this encoding scheme leads to the machine learning model giving more recent information more weight in the calculation of the probability of infection. The radial thickness of the area for each feature in a given day may be proportional to the time window for which the data point is valid. For each patient, the radius of the entire circle will be adjusted to the minimum radius under which the area condition holds true across all features included. The days with more data recorded will also have more bands and appear denser. This will depend on how many clinical features are measured (N_{f}) and their corresponding sample periods (t_{fi}). As a result, patients with more measurements will have a larger circle.

In step 2, people, equipment, and clinical environments that the patient comes into contact with are encoded in an image via slice-based encoding of information as shown in FIG. 2. The distinct contact events for the hospital unit across all patients may be tabulated into a table as they occur and assigned unique ID codes. Below is an example of such a table.

| Code | Event |
|---|---|
| 0 | Visit by nurse |
| 1 | Used equipment X |
| 2 | Visited facility A |
| 3 | Facility visit to surgery room |
| 4 | Facility visit to MRI room |

Compared to ring-based encoding of information in FIG. 1, the current encoding is another way of assigning feature importance to clinical measurements such as those shown in the table above. Because there is an incubation period for pathogens, it is not always true that the more recent encounter carries more weight in spreading the pathogen. As a result, each day (i.e., time unit) is treated equally as an equal division of the circle, i.e., each day has the same angular extent. For a given day, the duration the contact is proportional to the angle the corresponding pie includes. The radius of the slice representing each day is proportional to the total hours of contact the patient accumulated within the day. As a result, the area of each slice corresponds to the amount of contact associated with each event.

In step 3, based on the definition of each image described previously in steps 1 and 2, each of synthesized images is discretized to an image represented by H×W number of pixels. Image normalization and processing of missing data may also be performed at this time.

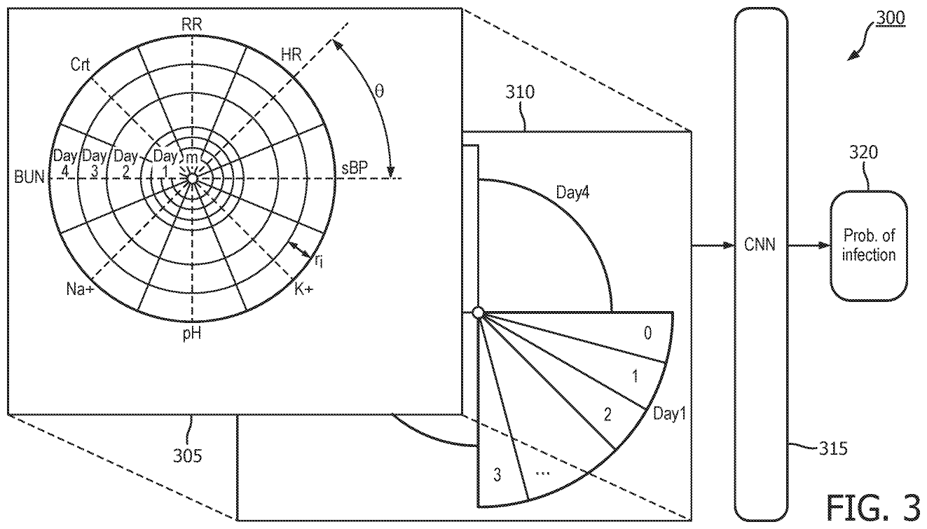

In step 4, the two synthetic images are input into a CNN to predict the risk of infection for each patient as a probability Pᵢ as shown in FIG. 3. In FIG. 3 the synthetic radar-chart image 305 of FIG. 1 and the synthetic slice-based image of FIG. 2 are shown as inputs to the CNN 315. The CNN 315 produces an output 320 of the intrinsic probability of infection Pᵢ for the patient.

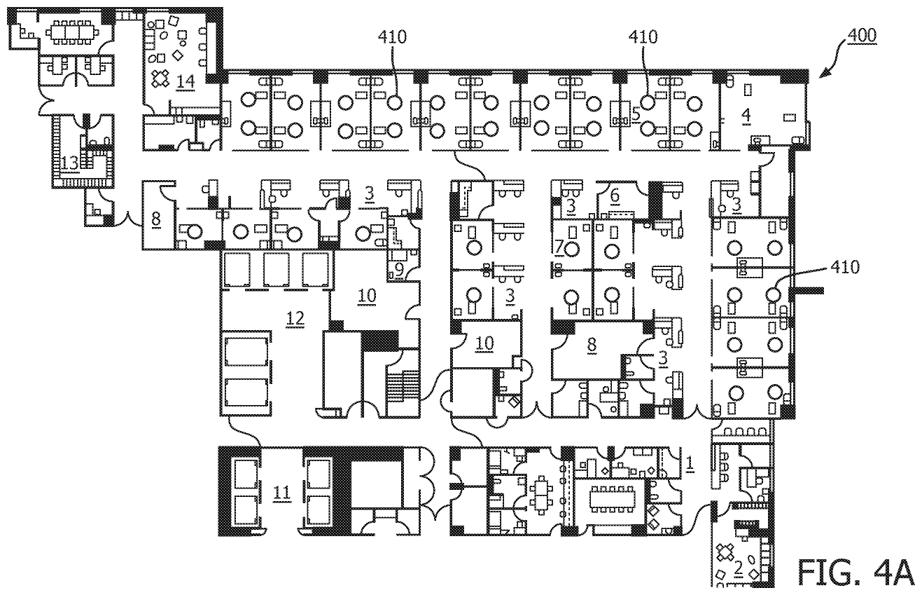

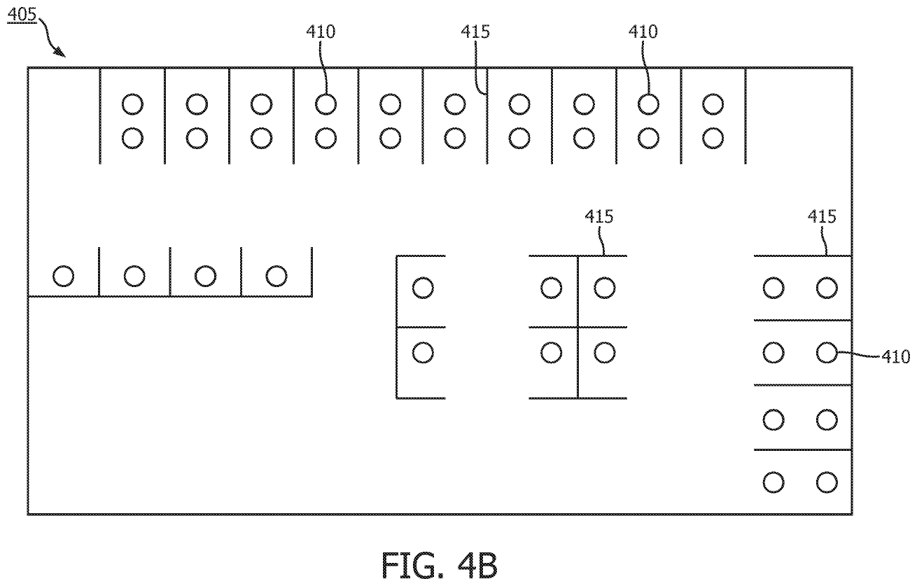

In step 5, the position of the patients within the entire clinical unit is transformed to a lattice representation. FIG. 4A illustrates an example floor layout of a unit in a hospital. In the floor layout 400, each patient bed is represented by a node 410. FIG. 4B illustrates a lattice representation of the floor layout. In the lattice representation 405, walls 415 separating patients 410 are denoted by lines, as isolation in general limits the spread of infections. Each side of the rectangular image for each room includes the walls without a door as these walls provide a barrier between patients. The floor layout 400 includes double and single rooms as shown.

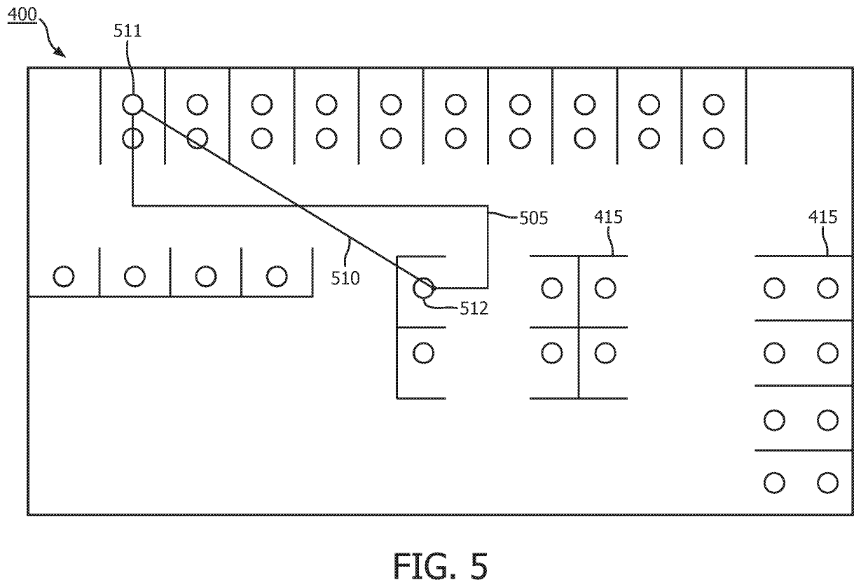

In step 6, a measure of similarity, S_{i,j}, is computed between patients i and j. This may be accomplished by a patient similarity measure based on features used in the generation of the images in steps 1 and 2. Alternatively, similarity may also be computed from state-of-the-art image recognition algorithm based on the synthetic images generated in step 3. Additional important features for similarity computation that have not been considered previously are metrics that represent physical distances between individual patients. FIG. 5 illustrates two different computations for the physical distance between two patients. The shortest distance 510 between patients 511 and 512 along a path 510 may be determined. Also, the distance between the patients 511 and 512 using a path 505 that extends only in the horizontal and vertical direction subject to the wall barriers may also be calculated. Distance information is important for infection prediction because it partially characterizes ease of infection transmission. In addition, the number of physical barriers that separate patients may be obtained for the shortest distance path 510 based upon the number walls 415 that the shortest path 510 crosses. These distances and number of barriers may also be used in the calculation of the similarity metric.

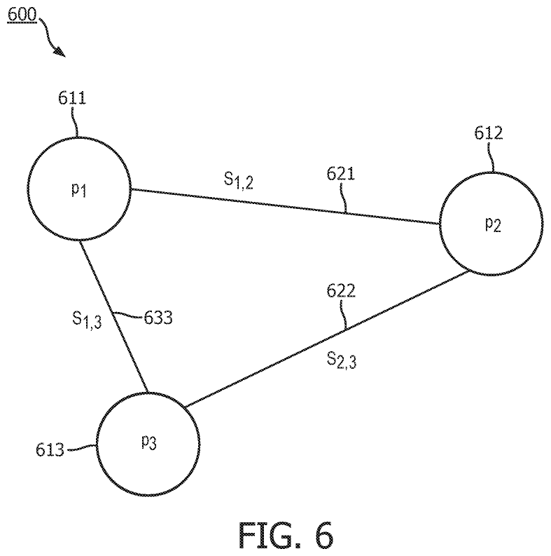

In step 7, the intrinsic risk of infection, Pᵢ determined in step 3 and patient similarity metrics determined in step 6 are formalized into a full-connected graphical model as shown in FIG. 6. FIG. 6 shows three nodes 611, 612, 613 where each node represents a single patient, and each node 611, 612, 613 will be assigned the intrinsic probability P₁, P₂, P₃ respectively. The edges 621, 622, 633 between nodes i and j are assigned patient similarity metrics, S_{1,2}, S_{2,3}, S_{1,3}, respectively, as weights. A node uses the infection risk of its neighbors to determine the final infection risk probability Pᵢ. This computation can be as simple as using the weighted sum of neighbors to adjust the intrinsic probability of infection for each patent. Alternatively, this can be a more complicated function modeling the spread of infection.

Additional considerations for the model may include the following. The data duration T and the sample period of each feature t_{fi} may be adjusted to the characteristics of the pathogen as well as the given physiological feature. For instance, the longer the incubation period the pathogen, the longer the data will be kept; vital signs are usually more frequently measured than labs and, therefore, are likely to have shorter sample periods. In the current method, the treatment patient receives for the infection (e.g., antibiotics) is not explicitly encoded. Instead, patient characteristics that reflect treatment responses from these interventions are included. The rationale is that interventions are only effective if patient recovers; otherwise, the intervention does not contribute to the severity or spread of infection.

Possible areas of application of the method described herein may include early prediction, risk stratification, and improved biomarker identification. Here, infection onset is identified by existing clinician annotations or definitive clinical markers (e.g., microbiology culture with 4+ days of antibiotic administration). The method described may be used for analysis of sepsis. More complex functions of physiology and interaction may be implemented for image generation in steps 1 and 2, such as adding weights to areas for known definitive biomarkers. Furthermore, an intensive care unit (ICU) may be the geographic entity. In fact, all hospital facilities that share similar recourses may be lumped together as one hospital unit for the model: for instance, several ICUs together, or a general ward and ICU if transfer between these units are frequent.

The implementation of the model described above focuses on the inpatient setting, where patients remain relatively stationary. As a result, the distance metrics are relatively simple and small in number. On the other hand, this model may be extended for the military or any other application, where people constantly move. This would need a more dynamic description of distance than described in FIG. 5. These distances and the surrounding environments may be recorded by radar devices, GPS systems, and/or any other available location systems. Distances between individuals may be updated according to distinct events performed by groups of individuals. Features describing the environment, such as air quality, radiation exposure, and altitude, may also be added to the feature maps.

Also, additional layers/image channels may be added to encode other categories of information. For instance, in the current implementation, the treatment patient receives for the infection (e.g., antibiotics) is not explicitly encoded, but can be included as needed. Pathogen information as they become available may also be added, although this may be later in the workflow. The following features may also be included in the image generation steps 2, 3, and 4:

Patient-Specific Information
physiology
vitals (heart rate, body surface temperature, respiratory rate, etc.)
biomarkers
e.g., C-Reactive protein, full blood count, procalcitonin, serology, gram stains, etc.)
e.g., interleukin
e.g., glucose, lactate, creatinine, blood urea
high-fidelity waveform data (ECG, ventilator waveform, heart sound, capnography, etc.)
for heart rate (e.g., heart rate variability (HRV), p-wave, QRS, etc. morphology,) and respiration characteristics (e.g., airway flow & resistance, pulse oximetry, etc.)
genomics of host-response to reflect infection-induced DNA damage and Modulation of DNA damage response.
gene micro-array data

Environment
radiation exposure
altitude
air pollutants
medical intervention for device-related infection
surgical procedures (ICD9 and CPT codes)
central line-associated bloodstream infections (CLABSI), ventilator-associated pneumonias (VAP), or urinary catheter-associated urinary tract infections (CAUTI)

Pathogen-Specific Information
sequence data: single nucleotide polymorphisms (SNAP)
for generation of phylogenetic tree and antibiograms

The methods described for transforming patient data into images may be easily generalized for other machine learning tasks than infection prediction.

The methods described for transforming patient data into images enable temporal data or time series into be input into a CNN without the need of aligning time points across different features via imputation.

The embodiments described herein solve the technological problem of predicting the transmission of infection between patients. The embodiments encode various patient data into synthetic images which are then processed using machine learning models to determine the probability of infection for each patient. Then the spatial layout of the facility is then used to determine a final probability infection for each patient based upon each patient's location relative to other patients. These various aspects of the embodiments allow for an accurate calculation of the probability of infection for each patient taking into account the layout of the facility and the locations of the various patients.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above including implementing the CNN and the generation and analysis of graphical model of the patients in the facility.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method of determining the infection risk probability for a patient, comprising:
encoding physiological data of the patient into a first synthetic image;
encoding environmental data of the patient into a second synthetic image;
determining an intrinsic probability of infection for the patient based upon the first synthetic image and the second synthetic image using a machine learning model;
determining patterns of infection transmission by generating a graphical model based upon the intrinsic probability of patients based upon similarity scores between the patient and the other patients; and
determining the infection risk probability for the patient based upon the graphical model and the intrinsic probability of infection for the patient and the other patients.

2. The method of claim 1, wherein the first synthetic image is a radar-type chart where each data parameter of the physiological data is encoded by an angle, the time and effective duration of the data parameter is encoded by the position and length of a segment on a radius, and the value of the data parameter is encoded as a gray scale value for the portion of the first synthetic image corresponding to the data parameter.

3. The method of claim 2, wherein the radius encoding of the data proceeds from the center of to the outer boundaries of the circle along the radius based upon the time of the data parameter from the earliest time to the most recent time.

4. The method of claim 1, wherein the second synthetic image is a circular slice-based image where each day includes the same angular extent and each environmental parameter is encoded as a slice of a day wherein the angular extent of the slice indicates the duration of the environmental parameter and the gray scale value of the slice indicated a code associated with the environmental parameter.

5. The method of claim 4, wherein the radius of the slice indicates the total duration of all environmental parameters for the day.

6. The method of claim 1, further comprising processing the first synthetic image and the second synthetic image into a predetermined number of image pixels with discrete values before determining an intrinsic probability of infection for the patient.

7. The method of claim 1, further comprising generating a lattice representation of the of the patient facility indicating the location of the patients in the facility and the barriers separating the patients.

8. The method of claim 7, wherein the graphical model includes a node for each patient and edges between each of the nodes indicating the similarity metric between each of the patients and wherein the graphical model is based upon the lattice representation.

9. The method of claim 8, wherein the similarity metric between two patients is based upon the first synthetic images and the second synthetic images of the two patients.

10. The method of claim 9, wherein the similarity metric between two patients is based upon the distance between the two patients based upon the lattice representation.

11. The method of claim 10, wherein the similarity metric between two patients is further based upon the barriers between the two patients.

12. The method of claim 1, wherein determining the infection risk probability for the patient is based on the weighted sum of the intrinsic probability of infection of the other patients where the similarity metrics are used as weights.

13. A non-transitory machine-readable storage medium encoded with instructions for deterring
the infection risk probability for a patient, comprising:
instructions for encoding physiological data of the patient into a first synthetic image;
instructions for encoding environmental data of the patient into a second synthetic image;
instructions for determining an intrinsic probability of infection for the patient based upon the first synthetic image and the second synthetic image using a machine learning model;
instructions for generating a graphical model based upon the patient and other patients based upon similarity scores between the patient and the other patients; and
instructions for determining the infection risk probability for the patient based upon the graphical model and the intrinsic probability of infection for the patient and the other patients.

14. The non-transitory machine-readable storage medium of claim 13, wherein the first synthetic image is a radar-type chart where each data parameter of the physiological data is encoded by an angle, the time and effective duration of the data parameter is encoded by the position and length of a segment on a radius, and the value of the data parameter is encoded as a gray scale value for the portion of the first synthetic image corresponding to the data parameter.

15. The non-transitory machine-readable storage medium of claim 14, wherein the radius encoding of the data proceeds from the center to the boundary of the circle based upon the time of the data parameter from the earliest time to the most recent time.

16. The non-transitory machine-readable storage medium of claim 13, wherein the second synthetic image is a circular slice-based image where each day includes the same angular extent and each environmental parameter is encoded as a slice of a day wherein the angular extent of the slice indicates the duration of the environmental parameter and the gray scale value of the slice indicated a code associated with the environmental parameter.

17. The non-transitory machine-readable storage medium of claim 16, wherein the radius of the slice indicates the total duration of all environmental parameters for the day.

18. The non-transitory machine-readable storage medium of claim 13, further comprising instructions for processing the first synthetic image and the second synthetic image into a predetermined number of image pixels with discrete values before determining an intrinsic probability of infection for the patient.

19. The non-transitory machine-readable storage medium of claim 13, further comprising instructions for generating a lattice representation of the of the patient facility indicating the location of the patients in the facility and the barriers separating the patients.

20. The non-transitory machine-readable storage medium of claim 19, wherein the graphical model includes a node for each patient and edges between each of the nodes indicating the similarity metric between each of the patients and wherein the graphical model is based upon the lattice representation.

21. The non-transitory machine-readable storage medium of claim 20, wherein the similarity metric between two patients is based upon the first synthetic images and the second synthetic images of the two patients.

22. The non-transitory machine-readable storage medium of claim 21, wherein the similarity metric between two patients is based upon the distance between the two patients based upon the lattice representation.

23. The non-transitory machine-readable storage medium of claim 22, wherein the similarity metric between two patients is further based upon the barriers between the two patients.

24. The non-transitory machine-readable storage medium of claim 13, wherein determining the infection risk probability for the patient is based on the weighted sum of the intrinsic probability of infection of the other patients where the similarity metrics are used as weights.
